SEQUENCE LISTING (6 sequences)

SEQ ID NO: 1 - protein, 4 aa, synthetic construct
  SITE 4: note = Residue attached to 2-(aminomethyl)-N,N'-didodecylbutanediamide
HHHH

SEQ ID NO: 2 - protein, 4 aa, synthetic construct
  SITE 4: note = Residue attached to 2-(aminomethyl)-N,N'-didodecylbutanediamide
KHHH

SEQ ID NO: 3 - DNA, 21 bp, synthetic construct
  source 1..21: mol_type = other DNA
  misc_feature 1..19: note = RNA
  misc_feature 20..21: note = DNA
cttacgctgagtacttcgatt

SEQ ID NO: 4 - DNA, 21 bp, synthetic construct
  source 1..21: mol_type = other DNA
  misc_feature 1..19: note = RNA
  misc_feature 20..21: note = DNA
ggagatctccgaaagctgctt

SEQ ID NO: 5 - DNA, 21 bp, synthetic construct
  source 1..21: mol_type = other DNA
  misc_feature 1..19: note = RNA
  misc_feature 20..21: note = DNA
ggtcttcaacactaccagctt

SEQ ID NO: 6 - DNA, 21 bp, synthetic construct
  source 1..21: mol_type = other DNA
  misc_feature 1..19: note = RNA
  misc_feature 20..21: note = DNA
ggtcttcaactcagccagctt

(19) United States
(12) Patent Application Publication
(10) Pub. No.: US 2026/0091126 A1
(43) Pub. Date: Apr. 2, 2026

(54) AMINO ACID-MODIFIED LIPIDS FOR RNA DELIVERY

(71) Applicant: The Curators of the University of Missouri, Columbia, MO (US)

(72) Inventors: Kun CHENG, Overland Park, KS (US); Pratikkumar Vinodchandra PATEL, Scarborough, ME (US)

(21) Appl. No.: 19/113,514

(22) PCT Filed: Sep. 19, 2023
(86) PCT No.: PCT/US2023/074552

§ 371 Date: Mar. 20, 2025

Related U.S. Application Data
(60) Provisional application No. 63/408,257, filed on Sep. 20, 2022.

(51) Int. Cl. A61K 47/69; A61K 47/54; A61K 47/64; C07K 5/068; C07K 5/078; C07K 5/09; C07K 5/097; C07K 5/11; C07K 5/117; C12N 15/88

(52) U.S. Cl. CPC A61K 47/6929; A61K 47/542; A61K 47/6455; C07K 5/06086; C07K 5/06147; C07K 5/0815; C07K 5/0821; C07K 5/1019; C07K 5/1024; C12N 15/88

(57) ABSTRACT

Described herein is the preparation and use of amino acid-modified lipids for delivery of a small interfering RNA (siRNA), mRNA, miRNA, shRNA, or oligonucleotide via formation of lipid nanoparticles (LNPs) comprising the siRNA, mRNA, miRNA, shRNA, or oligonucleotide and the amino acid-modified lipid. Use of the described lipid nanoparticles to silence overexpression of oncogenes is described.

### CROSS-REFERENCE TO RELATED APPLICATIONS

This application claims the benefit under 35 U.S.C § 119 (e) of U.S. Provisional Application No. 63/408,257 filed on Sep. 20, 2022, the entirety of the disclosure of which is incorporated herein by reference.

### GOVERNMENT RIGHTS

This invention was made with government support under R01GM121798, R01CA231099, and R01AA021510 awarded by the National Institutes of Health. The government has certain rights in the invention.

### TECHNICAL FIELD

The invention described herein pertains to the preparation and use of a new class of amino acid-modified lipids for delivery of small interfering RNAs (siRNAs), mRNA, miRNA, shRNA, or oligonucleotides.

### BACKGROUND

Since the first report of RNA interference (RNAi) in 1998, small interfering RNAs (siRNAs) have been investigated in the treatment of a wide range of human diseases including genetic disorders, viral infections, autoimmune diseases, and cancers. Once siRNAs reach the cytoplasm, they are incorporated into the RNA-induced silencing complex (RISC), which binds to the targeted mRNA sequence and cleaves it, thereby silencing the targeted gene [1]. However, siRNAs are highly prone to degradation in biological systems and inefficient for transport through the cell membrane due to their size and polyanionic structure. Advances in the development of biocompatible materials for delivering and enhancing the efficacy of siRNAs have promoted the field of RNA therapeutics [2].

Nonviral delivery vehicles are the most promising for siRNA delivery because they present very little to no immunogenic response [3]. Among them, ionizable amine-bearing lipid nanoparticles (LNPs) are a highly advanced platform for nucleic acid delivery [4]. LNPs with ionizable amines are effectively protonated during endosomal maturation and bind with the endosomal membrane and enhance buffering capacity [5]. LNPs with appropriate structure and effective ionization promote endosomal release to deliver siRNAs into the cytoplasm [6]. However, even highly effective lipids or polymers can only release a very small fraction of siRNAs into the cytoplasm, while the majority of siRNAs are trapped inside the endosome. Thus, endosomal escape is the rate-limiting step of siRNA delivery, and its efficacy can be enhanced by discovering new effective materials [5, 7].

Various lipids have been developed for nucleic acid delivery by large library-based screening or rational design-based approaches [8]. The recent FDA-approved LNP-based siRNA therapy Onpattro (patisiran) was developed using the ionizable lipid Dlin-MC3-DMA (MC3). The LNPs of MC3 bind with apolipoprotein E (APOE) in the circulation, which leads to high uptake in liver cells through low-density lipoprotein receptors. However, the low biodegradability and unfavorable adverse effects of MC3 LNPs hinder their use in chronic therapies [9]. The biocompatibility of lipids could be improved by introducing biodegradability, which may facilitate the elimination of lipids once they performed their designed task of siRNA delivery to the appropriate intracellular compartments [10]. In addition, environment-responsive ionizable lipids can reduce the toxicity associated with cationic lipids and improve clinical applications [11].

Despite the success of siRNA delivery to the liver, non-hepatic siRNA delivery for cancer therapy remains a huge challenge. As a result, new strategies and approaches are needed to develop siRNA-based cancer therapies [12]. In addition, nanoparticles should be biodegradable and safe for frequently repeated doses. Previously, highly active siRNAs for IKKα and IKBKE were discovered and their promising activity in inhibiting the invasiveness of prostate cancer and breast cancer, respectively demonstrated [13, 14]. In addition, a cholesteryl peptide-based system was developed to deliver siRNAs to the liver, pancreatic cancer, and breast cancer [15-17].

### SUMMARY

A new class of amino acid-modified lipids to deliver RNAs to various types of cancer cells is described herein. The headgroup of the lipids is modified with histidine or an analog thereof and/or lysine or an analog thereof. LNPs made from these lipids exhibit effective endosomal escape to deliver an RNA in various cancer cells and exhibit significant silencing activity. Moreover, the in vivo activity of the amino acid-modified LNPs was evaluated in a pancreatic cancer mouse model.

In one embodiment of the disclosure a compound having formula I

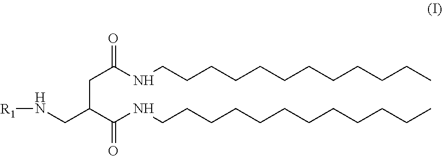

or a solvate, hydrate, or salt thereof;
wherein R₁ is a peptide of sequence (AA₁)ₐ-(AA₂)_{b} connected via the C-terminus of the peptide to the lipid moiety

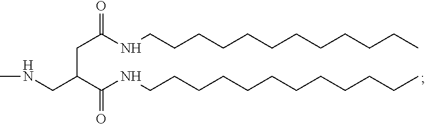 [SMILES: CCCCCCCCCCCCNC(=O)CC(CNC)C(=O)NCCCCCCCCCCCC]

AA₁ is selected from the group consisting of 2,3-diaminopropanoic acid, 2,4-diaminobutanoic acid, ornithine, lysine or an analog thereof, 2,7-heptanoic acid, 2,8-octanoic acid, 2-amino-3-guanidinopropanoic acid, 2-amino-4-guanidino-butanoic acid and 2-amino-6-guanidino pentanoic acid, and arginine, each of which can be in the 2R, 2S, or racemic form;
AA₂ is histidine or an analog thereof in the 2R, 2S, or racemic form; and
a is 0 or 1 and b is 0, 1, 2, 3, or 4, where at least one of a or b is not 0.

In another embodiment of the disclosure, a liquid nanoparticle (LNP) comprising the compound of formula I is disclosed.

In another embodiment of the disclosure, the LNP described above further comprises one or RNAs.

In another embodiment of the disclosure, use of the LNP described above to treat a patient in need of relief from a cancer is described.

### BRIEF DESCRIPTION OF THE DRAWINGS

FIG. 1. Schematics of the lipid nanoparticle and cellular uptake in cancer cell. Amino acid-modified lipids encapsulate siRNAs and are internalized with cells. Ionizable LNPs protonated in the acidic endosome and produce the endosomal escape by interacting with phospholipids or proton sponge effect, to deliver the siRNAs into the cytoplasm.

FIG. 2. List of synthesized ionizable lipids. Lysine and histidine were used in different ratios to modify the lipid headgroup. Figure discloses SEQ ID NOS 1 and 2, respectively, in order of appearance.

FIG. 3. Characterization of the lipid nanoparticles. Particle size (A), polydispersity index (B), and zeta potential (C) of LNPs were measured using a Zetasizer. All LNPs were evaluated for siRNA entrapment by gel retardation assay (D). All results are presented as mean±SD (n=3).

FIG. 4. Luciferase silencing and cell cytotoxicity of the lipid nanoparticles. (A) Luciferase levels of the cells treated with luciferase siRNA LNPs were normalized to the cells treated with scramble siRNA LNPs. (B) Cell viability was measured at 24 h and 48 h post-transfection. (C) The apparent pKa of LNPs was measured by plotting the TNS fluorescence against pH values. All results are presented as mean±SD (n=3). (D) The plot of pKa versus residual luciferase. (E) TNS fluorescence at late endosomal pH 5.2.

FIG. 5. Morphology, toxicity, hemolysis, and serum stability of the LHHK LNPs. (A) TEM images of LHHK LNPs. Scale bars represent 200 nm. (B) Toxicity of LHHK LNPs and Lipofectamine-2000. (C) Hemolysis assay of LHHK LNPs at pH 7.4 and pH 5.6. (D) Serum stability of LHHK LNPs in 50% human serum for 0, 1, 6, and 24 h. All experiments were carried out in triplicate. All results are presented as mean±SD (n=3).

FIG. 6. Cellular uptake of the LNPs in PC3 cells. (A) Flowcytometry analysis of PC3 cells incubated with LNPs encapsulating Cy5-labeled IKKa siRNA. Percentage of Cy5-labeled cells (B) and fluorescence intensity of PC3 cells (C) at 1, 2, and 4 h post-transfection. Confocal microscopy images of PC3 cells treated with LNPs at 2 h (D) and 4 h (E) post-transfection. (F) Co-localization of Cy5-siRNA (red) and lysosome (green) in cells at 4 h post-transfection was analyzed as the Pearson's correlation coefficient using ImageJ. Scale bars represent 20 μm.

FIG. 7. Gene silencing and anti-proliferation effect of LHHK LNPs. The human IKKa siRNA-loaded LNPs of LHHK and LHHHK were examined against the scrambled siRNA-loaded LNPs. The IKKa mRNA level was measured in PC3 (A) and DU145 (B) cells after 24 h of transfection. (C) Similarly, the human IKBKE siRNA and mouse IKBKE siRNA-loaded LHHK LNPs were examined. The IKBKE mRNA level was measured in PANC-1 cells and PANC02 cells after 24 h of transfection. (D) Cell proliferation of PANC-1 and PANC02 cells was evaluated after 96 h of transfection.

FIG. 8. In vivo antitumor activity in a pancreatic cancer mouse model. IKBKE siRNA and scrambled siRNA loaded LNPs of LHHK were evaluated for the effect and safety of IKBKE siRNA treatment with LHHK LNPs for the PANC02 tumor based murine model of pancreatic cancer. (A) LNPs were administrated around the tumor at a dose of 0.4 mg/kg siRNA on days 6, 9, 12 and 15. (B) Tumor growth curve. Results are presented as mean±SEM (n=7). (C) Tumor weight. Results are presented as mean±SEM (n=7). (D) Normalize body weight. Results are presented as mean±SD (n=7). (E) Biochemical analysis of the plasma. Results are presented as mean±SD (n=7).

### DETAILED DESCRIPTION

While the concepts of the present disclosure are illustrated and described in detail in the description herein, results in the description are to be considered as exemplary and not restrictive in character; it being understood that only illustrative embodiments are shown and described and that all changes and modifications that come within the spirit of the disclosure are desired to be protected.

The following clauses describe several non-limiting embodiments of the disclosure.

1. A compound having formula I

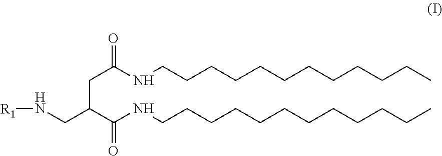

or a solvate, hydrate, or salt thereof;
wherein R₁ is a peptide of sequence (AA₁)ₐ-(AA₂)_{b} connected via the C-terminus of the peptide to the lipid moiety

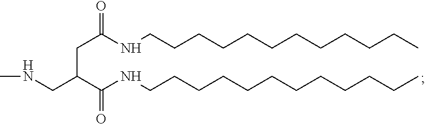 [SMILES: CCCCCCCCCCCCNC(=O)CC(CNC)C(=O)NCCCCCCCCCCCC]

AA₁ is selected from the group consisting of 2,3-diaminopropanoic acid, 2,4-diaminobutanoic acid, ornithine, lysine or an analog thereof, 2,7-heptanoic acid, 2,8-octanoic acid, 2-amino-3-guanidinopropanoic acid, 2-amino-4-guanidino-butanoic acid and 2-amino-6-guanidino pentanoic acid, and arginine or an analog thereof, each of which can be in the 2R, 2S, or racemic form;
AA₂ is histidine or an analog thereof in the 2R, 2S, or racemic form; and
a is 0 or 1 and b is 0, 1, 2, 3, or 4, where at least one of a or b is not 0.

2. The compound of clause 1, or a solvate, hydrate, or salt thereof, wherein a is 0.

3. The compound of clause 1, or a solvate, hydrate, or salt thereof, wherein a is 1.

4. The compound of clause 3, or a solvate, hydrate, or salt thereof, wherein b is 0.

5. The compound of clause 2, or a solvate, hydrate, or salt thereof, wherein b is 1, 2, 3, or 4.

6. The compound of clause 3, or a solvate, hydrate, or salt thereof, where b is 1, 2, or 3.

7. The compound of any one of clauses 1 to 6, or a solvate, hydrate, or salt thereof, wherein AA₁ is lysine.

8. The compound of any one of clauses 1 to 6, or a solvate, hydrate, or salt thereof, wherein AA₂ is histidine.

9. The compound of clause any one of the preceding clauses, or a solvate, hydrate, or salt thereof, wherein AA₁ is lysine and AA₂ is histidine.

10. The compound of any one of the preceding clauses, or a solvate, hydrate, or salt thereof, wherein AA₁ is 2S-lysine.

11. The compound of any one of the preceding clauses, or a solvate, hydrate, or salt thereof, wherein AA₂ is 2S-histidine.

12. The compound of any one of the preceding clauses, or a solvate, hydrate, or salt thereof, wherein AA₁ is 2S-lysine and AA₂ is 2S-histidine.

13. The compound of any one of the preceding clauses, or a solvate, hydrate, or salt thereof, wherein the compound selected from the group consisting of

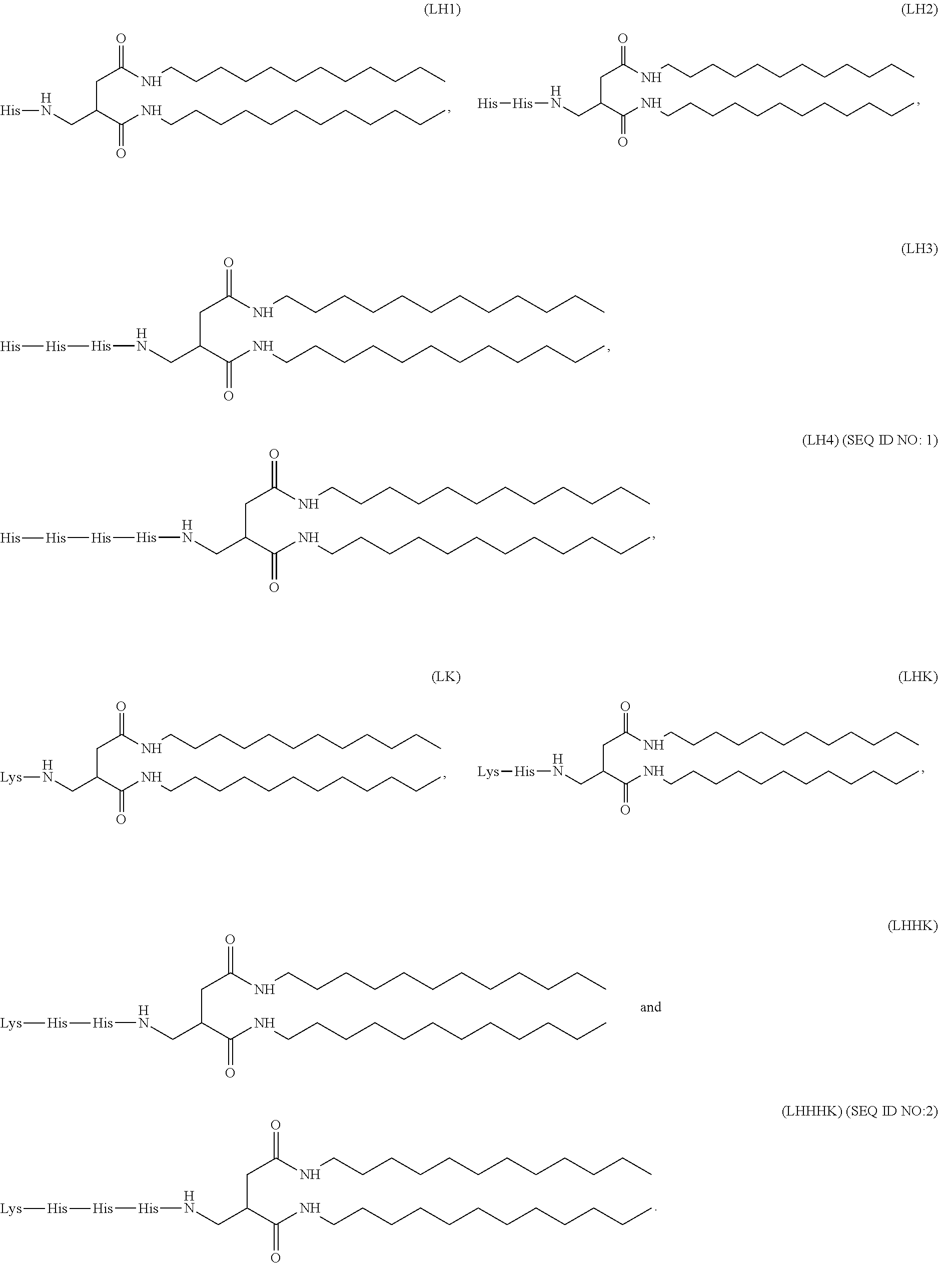

14. The compound of any one of preceding clauses, or a solvate, hydrate, or salt thereof, having formula

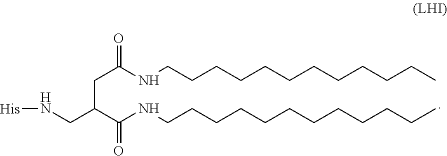

15. The compound of any one of clauses 1 to 13, or a solvate, hydrate, or salt thereof, having formula

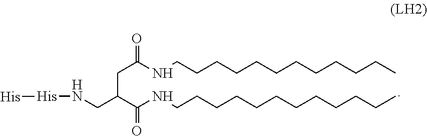

16. The compound of any one of clauses 1 to 13, or a solvate, hydrate, or salt thereof, having formula

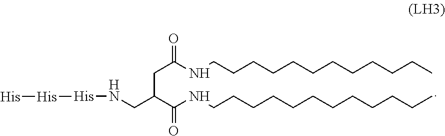

17. The compound of any one of clauses 1 to 13, or a solvate, hydrate, or salt thereof, having formula

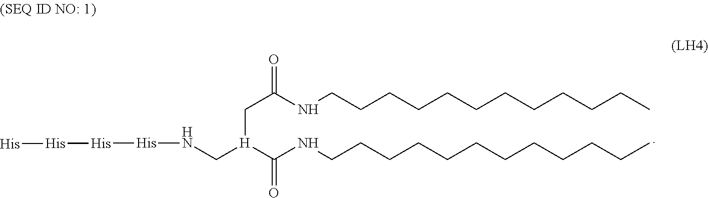

18. The compound of any one of clauses 1 to 13, or a solvate, hydrate, or salt thereof, having formula

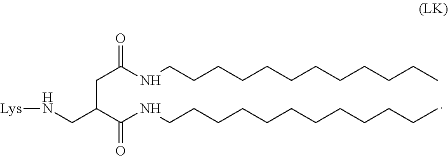

19. The compound of any one of clauses 1 to 13, or a solvate, hydrate, or salt thereof, having formula

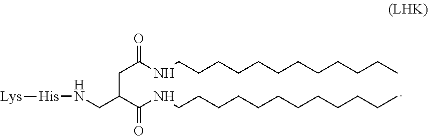

20. The compound of any one of clauses 1 to 13, or a solvate, hydrate, or salt thereof, having formula

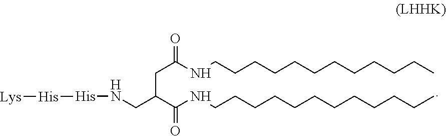

21. The compound of any one of clauses 1 to 13, or a solvate, hydrate, or salt thereof, having formula

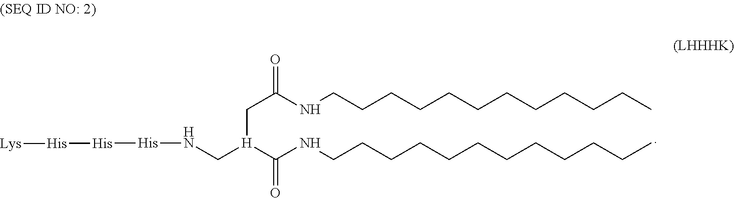

22. A lipid nanoparticle comprising one or more of the compounds of any one of the preceding clauses.

23. The lipid nanoparticle of clause 22 wherein a is 0.

24. The lipid nanoparticle of clause 22 wherein a is 1.

25. The lipid nanoparticle of clause 22 wherein b is 0.

26. The lipid nanoparticle of clause 23 wherein b is 1, 2, 3, or 4.

27. The lipid nanoparticle of clause 24 where b is 1, 2, or 3.

28 The lipid nanoparticle of any one of clauses 22 to 27 wherein AA₁ is lysine.

29 The lipid nanoparticle of any one of clauses 22 to 27 wherein AA₂ is histidine.

30. The lipid nanoparticle of any one of the preceding lipid nanoparticle clauses, wherein AA is lysine and AA₂ is histidine.

31. The lipid nanoparticle of any one of the preceding lipid nanoparticle clauses, wherein AA₁ is 2S-lysine.

32. The lipid nanoparticle of any one of the preceding lipid nanoparticle clauses, 21 wherein AA₂ is 2S-histidine.

33. The lipid nanoparticle of any one of the preceding lipid nanoparticle clauses, wherein AA₁ is 2S-lysine and AA₂ is 2S-histidine.

34. The lipid nanoparticle of any one of the preceding lipid nanoparticle clauses, wherein the compound is selected from the group consisting of

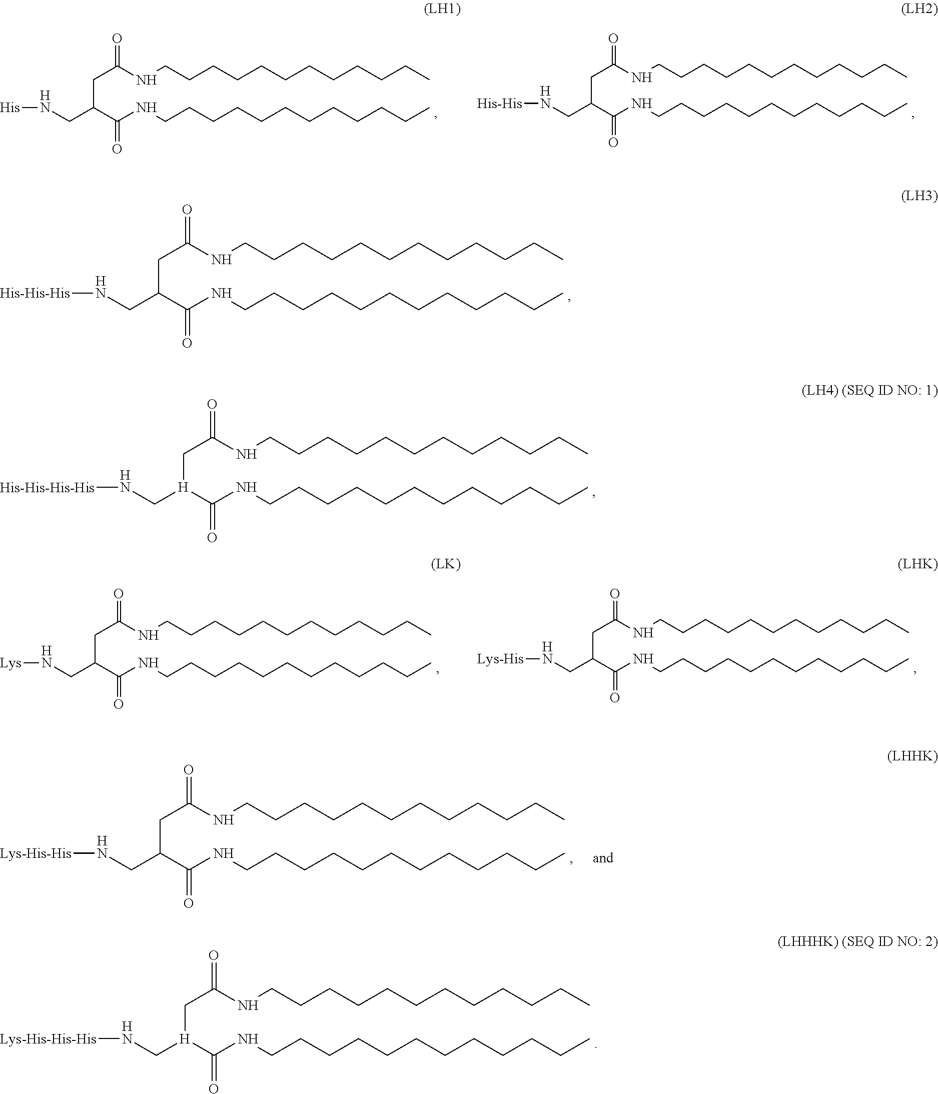

35. The lipid nanoparticle of any one of the preceding lipid nanoparticle clauses, wherein the compound has the formula

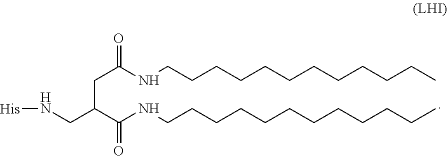

36. The lipid nanoparticle of any one of the preceding lipid nanoparticle clauses, wherein the compound has the formula

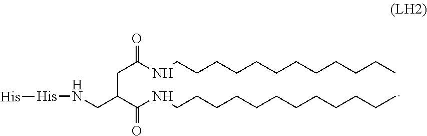

37. The lipid nanoparticle of any one of the preceding lipid nanoparticle clauses, wherein the compound has the formula

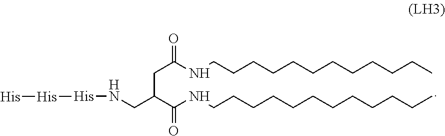

38. The lipid nanoparticle of any one of the preceding lipid nanoparticle clauses, wherein the compound has the formula

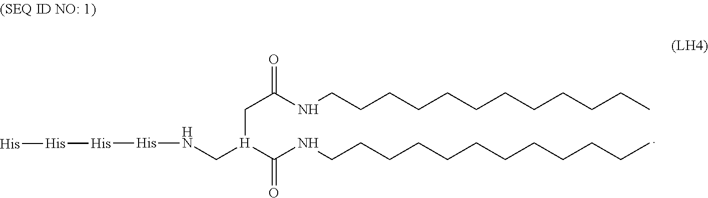

39. The lipid nanoparticle of any one of the preceding lipid nanoparticle clauses, wherein the compound has the formula

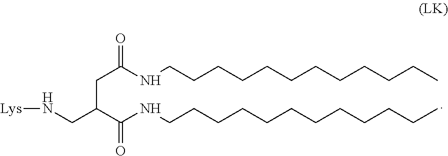

40. The lipid nanoparticle of any one of the preceding lipid nanoparticle clauses, wherein the compound has the formula

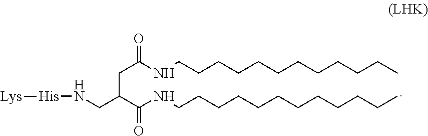

41. The lipid nanoparticle of any one of the preceding lipid nanoparticle clauses, wherein the compound has the formula

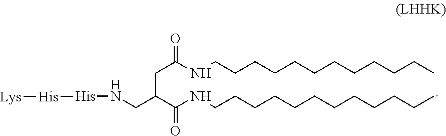

42. The lipid nanoparticle of any one of the preceding lipid nanoparticle clauses, wherein the compound has the formula

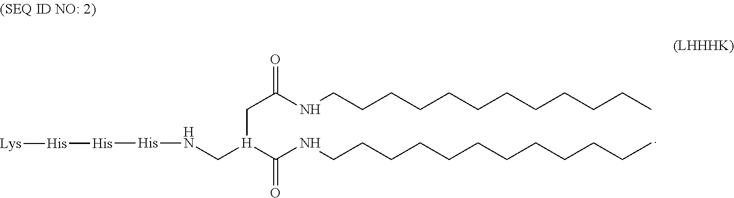

43. The lipid nanoparticle any one of the preceding lipid nanoparticle clauses, further comprising an RNA selected from the group consisting of siRNA, miRNA, shRNA, and mRNA.

44. The lipid nanoparticle of clause 43 wherein the RNA is a siRNA.

45. The lipid nanoparticle of clause 44 wherein the siRNA silences expression of an overexpressed oncogene related to the progression of a cancer, such as oncogenes IKKα and IKBKE.

46. The lipid nanoparticle of clause 45 wherein the cancer is selected from breast, prostate, or pancreatic cancer.

47. Use of the LNP of clause 45 to treat a patient in need of relief from the cancer.

As used herein an analog of a compound, is a compound having a structure similar to that of the compound, but differing from it in respect to a certain component. It can differ in one or more atoms, functional groups, or substructures, which are replaced with other atoms, groups, or substructures. For example:

An analog of lysine (c is 0) or arginine (c is 1) may have the following structure

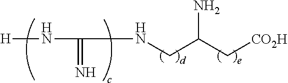

where d is an integer from 1 to 8, and e is 1 or 0.

An analog of histidine may have the following structure

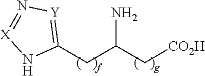

where X and Y are independently N or CH, f is 1 to 4, and g is 0 or 1.

siRNA-based treatment targeting tumorigenic genes (oncogenes) is a very promising approach for cancer therapy. To date, various genes that are overexpressed in cancerous cells and play a critical role in cancer progression have been identified, and many efforts have been made to develop siRNAs targeting these genes. Although the FDA has approved three siRNA therapies, all of them deliver siRNAs to the liver for the treatment of diseases related to liver gene malfunction. siRNA delivery to cancer cells remains a major challenge, and further improvement in the siRNA delivery system is required to harness the potential of siRNA-based cancer therapy [18].

Lipid nanoparticle is the most successful drug delivery system for RNA delivery. For example, MC3 lipid was used in ONPATTRO® (patisiran), the first-ever FDA-approved siRNA therapy. To enhance the tolerability of lipid nanoparticles, ester linkages are included to increase biodegradability without compromising efficacy. SM-102 and ALC-0135 have better biodegradability and pharmacokinetics than MC3 and were used to develop COVID-19 vaccines mRNA-1273 and BNT162b, respectively [19]. Various efforts have been invested in the development of biodegradable lipids with ester and disulfide motifs [20-23]. Biodegradable lipids were also developed using amide linkage for a combinatorial library [24, 25], and the best LNPs share several similarities in the structure including amide linkage [24]. Amino acids are the primary structural unit of proteins, and the introduction of amino acids in the structural modification of natural products and derivatives enhances their performance and minimizes the adverse effect [26]. Histidine and Lysine are essential amino acids and are considered safe for human consumption [27]. Previously, cholesteryl-peptides using histidine and lysine were developed and used in siRNA delivery for cancer treatment [16, 17]. Herein, are developed amino acid-modified lipids with multiple amide linkages to increase biodegradability and safety.

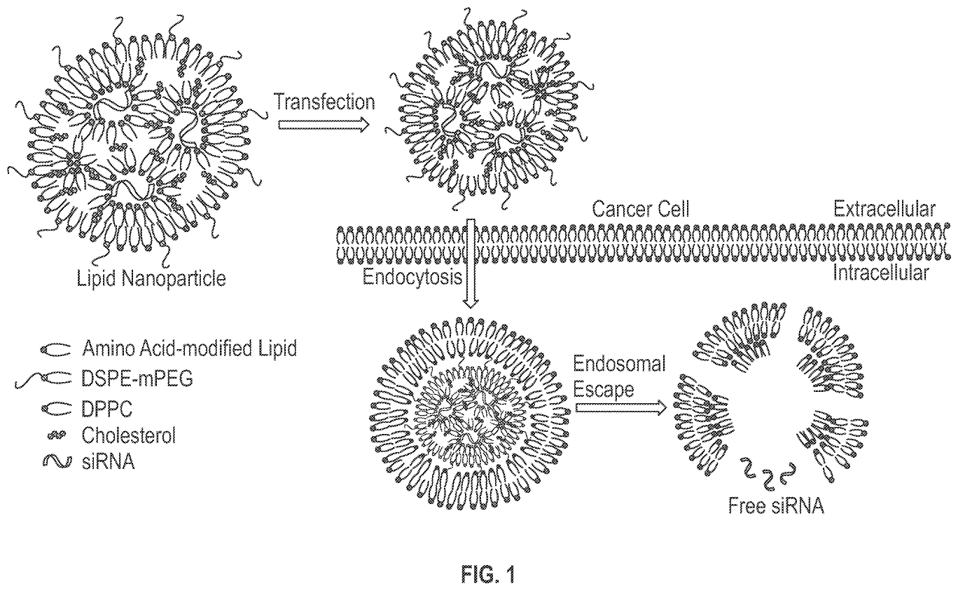

The objective was to develop biodegradable lipids for siRNA delivery. The biodegradability and toxicity were considered in designing the lipids. Cationic natural amino acids, such as lysine and histidine, are highly biocompatible and suitable candidates for headgroup modification in lipids. Therefore, a new class of amino acid-modified lipids including histidine and/or lysine was designed to demonstrate the intracellular siRNA delivery (FIG. 1) for anti-tumor activity.

### Synthesis of Amino Acid-Modified Lipids

The amino acid-modified lipids were developed by using the linker 2-(aminomethyl)butanedioic acid hydrochloride (Scheme 1), which contains one amine and two carboxylate groups. The amine group was protected using BOC anhydride (Intermediate A), and the two carboxylate groups were reacted with dodecyl amine to generate tails of the lipid in ‘Intermediate B’ by forming amide linkages. A ‘12 carbon’ in the lipid tail was used because a number of previous structural activity relationship studies suggested that lipids with 12 carbon tails yield the highest activity [28, 29]. Also, it was reported that lipid with amide linkages performed better for siRNA delivery in a combinatorial library screening study [24]. Later, ‘Intermediate C’ was prepared by Boc deprotection of ‘Intermediate B’ to make headgroup modifications. The C-termini of histidine and lysine were reacted with the free amine of ‘Intermediate C’.

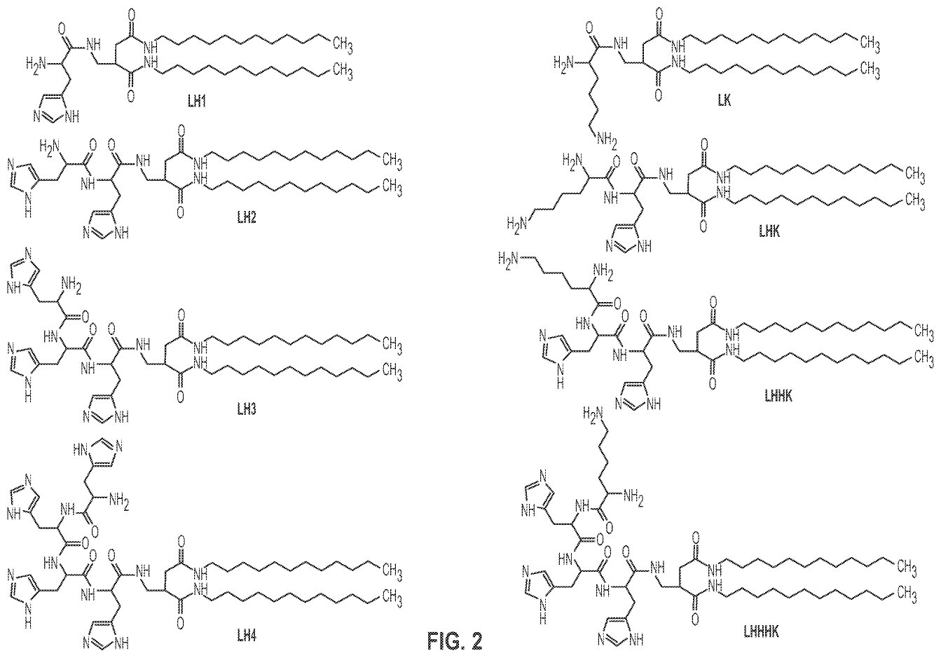

Eight lipid molecules were prepared by reacting lysine and histidine multiple times for headgroup modification (FIG. 2). All the synthesized lipids were characterized by 1HNMR and mass spectrometry methods (see supporting information). 1HNMR analysis of all the lipids confirmed the structure and high purity of the synthesized lipids. Proton peaks of the aromatic region (7-9) showed a clear trend with the number of histidine residues in the lipids. The mass analysis showed the M+1 peak of the corresponding molecular weight for lipids. All the synthesized lipid molecules had multiple amide linkages in the structure, which makes them suitable candidates for biodegradability and accelerated clearance in vivo.

Development and Characterization of siRNA Loaded LNPs

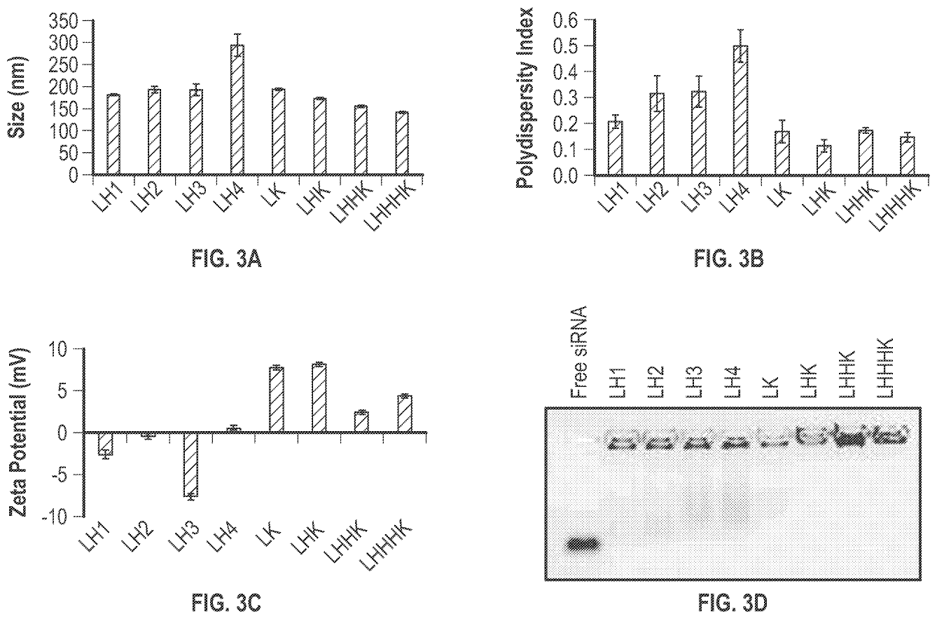

LNPs were prepared by mixing at a 7:1 (w/w) ratio of cationic lipid to siRNA. A dynamic light scattering analysis of the readily formed LNPs showed that their sizes ranged from 150-200 nm (FIG. 3A). Nanoparticles with a size of 100-200 nm are able to accumulate in tumors by the enhanced permeability and retention (EPR) effect [30].

Lipids with a terminal lysine have low PDI values compared to lipids with a terminal histidine headgroup (FIG. 3B). Nanoparticles with histidine at the end of the headgroup aggregated over time and showed increases in size and PDI after 24 h (Table S2). These lipids were positively charged at pH 3 and formed LNPs with siRNAs. However, after dialysis against PBS (pH 7.4), the ionizable lipids were deprotonated, and the bulky head of histidine led to the aggregation of LNPs. As the numbers of histidine in the headgroup increases, the aggregation of LNPs becomes more significant. By contrast, the lipids with lysine at the end of the headgroup formed stable nanoparticles, and the PDI values were below 0.2. In addition, the LNPs with positive zeta potentials between 0 to 10 (FIG. 3C) suggested that low charge-related toxicity will be associated with these LNPs. The lipids with terminal histidine headgroups had negative zeta potential values. This explains why the ionizable amines were in the deprotonated form at physiological pH, which led to the aggregation of LNPs. Moreover, the gel retardation assay suggested that all the LNPs efficiently entrap siRNAs (FIG. 3D).

### Preliminary Screening of LNPs for Luciferase Silencing and Cytotoxicity

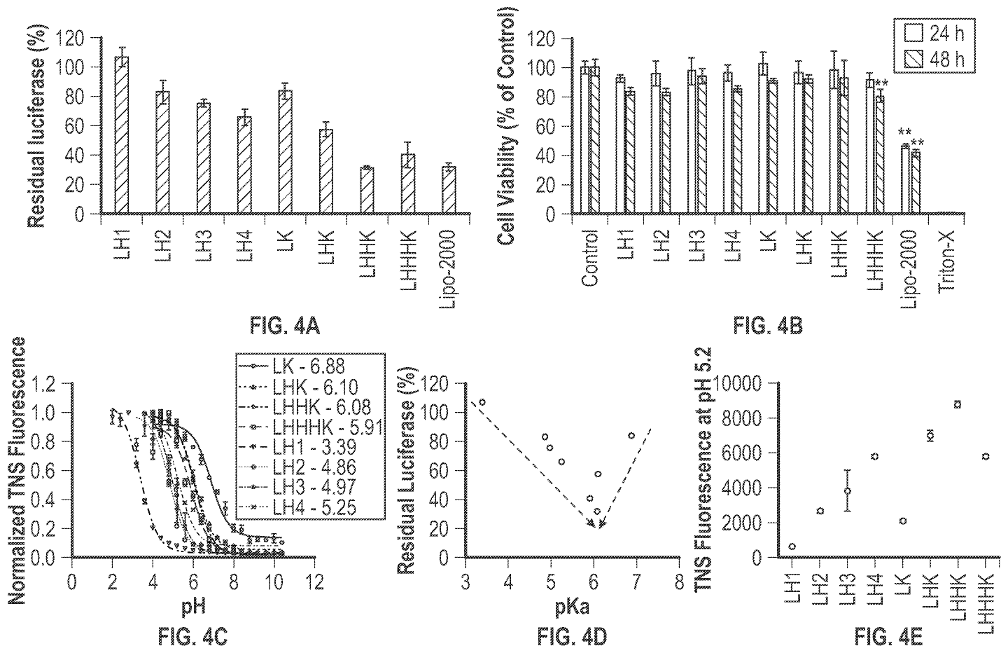

The silencing activity of the LNPs was evaluated in PC3-Luc cells using luciferase siRNA against scrambled siRNA (FIG. 4A). The LHHK LNPs achieved ˜70% luciferase silencing, which is similar as that of Lipofectamine-2000. The silencing activity of LNPs was improved with an increasing number of histidine residues (LH1 to LH4, LK to LHHK) in the head group. The LNPs of lipid LHHHK produced more than 50% luciferase silencing, which is the second most active lipid after LHHK.

The toxicity of the LNPs was measured using a cell viability assay (FIG. 4B), which was performed after 24 and 48 h of transfection. All lipids were relatively safe compared to Lipofectamine-2000 and exhibited very low cytotoxicity. These amino acid-modified and multiple amide bond-bearing lipids are highly biocompatible and biodegradable and thus produce significantly less cytotoxicity in the cells. The highly active LHHK LNPs produced relatively less cytotoxicity than Lipofectamine-2000 with a similar level of gene silencing.

### Effect of PKA on Gene Silencing

The pKa of all the LNPs was measured with a TNS fluorescence assay. As illustrated in FIG. 4C, the pKa values of the LNPs were in the range of 3.39-6.88, and they were correlated with the zeta potential of the LNPs. The LHHK LNP has a pKa of 6.08, which is in the ideal pKa range of 6-7 and capable of inducing effective endosomal escape [31]. In addition, luciferase silencing was correlated with the pKa in PC3-Luc cells. The silencing activity increased as the pKa value increased from 3.9 to 6, and the maximum gene silencing was found near pKa˜6 (FIG. 4D). Additionally, LNPs with low stability have pKa values below 5.5. These results are consistent with previous studies that demonstrated correlations among the physicochemical properties, stability, and gene silencing activity of LNPs [28, 32]. The TNS fluorescence of LNPs at pH 5.2 was analyzed to evaluate the degree of ionization at late endosomal pH (FIG. 4E). The results are consistent with the silencing activity (FIG. 4A) and suggest that LHHK LNP generate the highest degree of ionization during the endosomal maturation process.

### Characterization of LHHK LNPs for Morphology, Toxicity, Serum Stability, and Hemolytic Assay

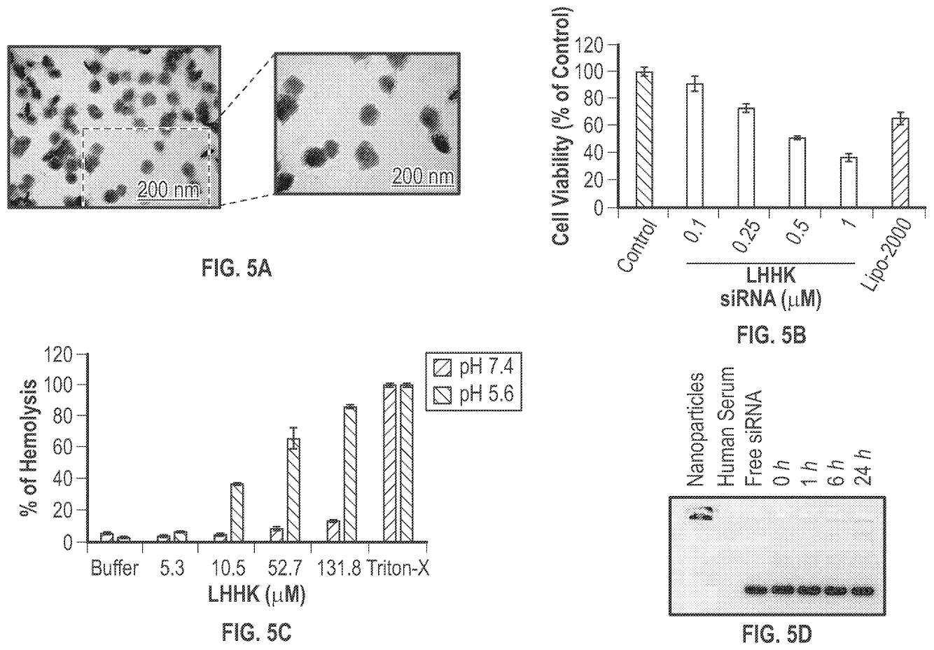

The LHHK LNPs were further evaluated by TEM, toxicity, hemolysis, and serum stability assays. The TEM analysis revealed that the LNPs were spherical in shape and approximately 150-200 nm in size (FIG. 5A). The toxicity of the LHHK LNPs was measured with various concentrations of siRNAs from 100 nM to 1 μM. The cytotoxicity of LHHK LNPs increased with the higher dose, however higher doses of LHHK LNPs were less cytotoxic compared to Lipofectamine-2000 at some extent (FIG. 5B). Subsequently, pH-sensitive endosomal disruption was evaluated with an ex vivo hemolysis assay [33]. The blank nanoparticles with LHHK concentrations of 5.3, 10.5, 26.4, and 65.9 μM are correspondence to the siRNA dose of 50 nM, 100 nM, 500 nM, and 1.25 μM. At pH 7.4, significant hemolysis was not observed even at high LNP concentrations, suggesting the safety of LNPs at physiological pH (FIG. 5C). At pH 5.6 (below pKa), LNPs produce hemolysis, and the effect is increased as the concentration increases, which explains why the LNPs could promote endosomal escape for intracellular siRNA delivery. In addition, the siRNA stability in serum was evaluated with agarose gel electrophoresis. The LHHK LNPs protected siRNAs in 50% human serum up to 24 h (FIG. 5D).

Cellular Uptake of LNPs for Intracellular siRNA Delivery

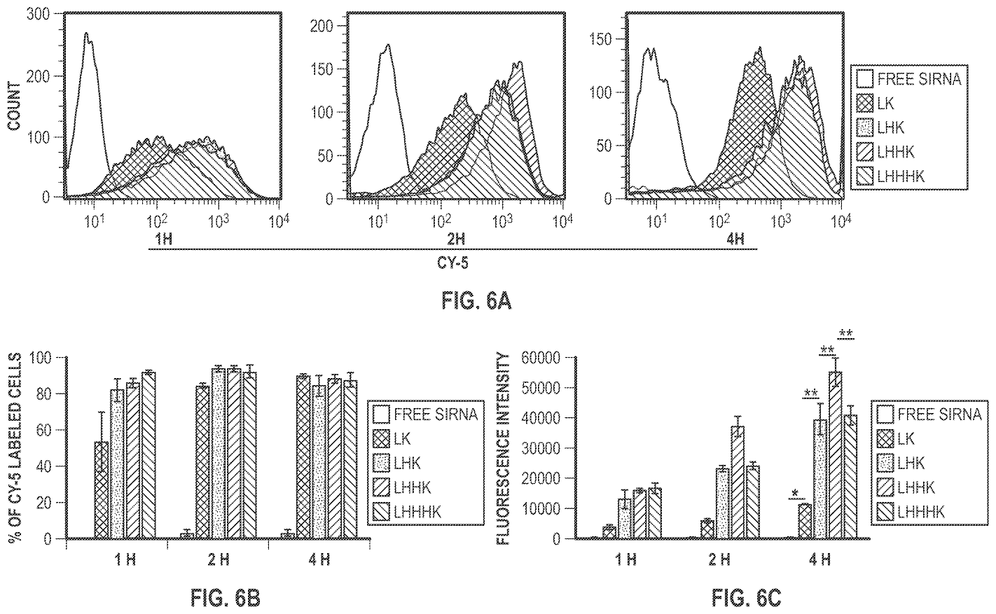

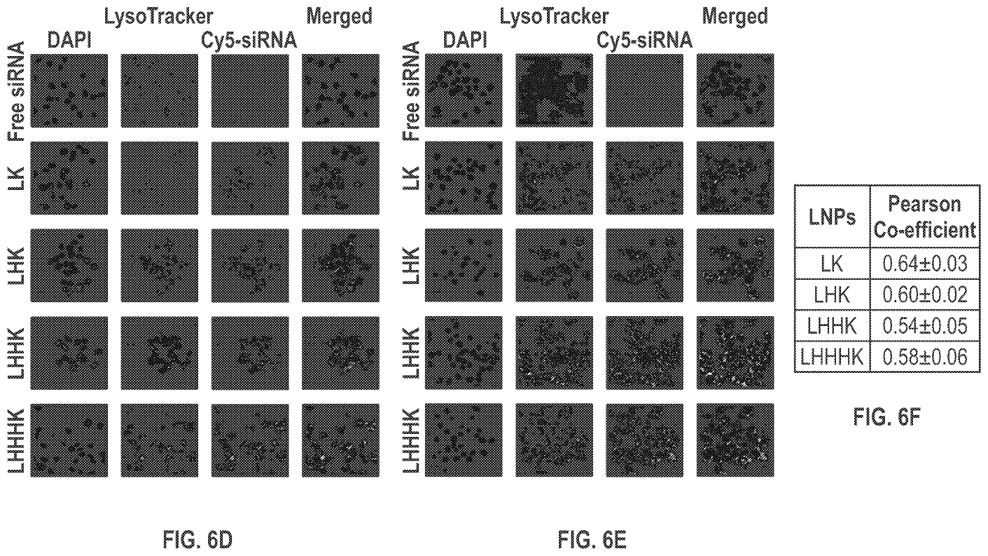

To understand the higher activity of the lipid LHHK, four lysine-modified lipids (LK, LHK, LHHK, LHHHK) were evaluated for intracellular siRNA delivery in PC3 cells using flow cytometry and confocal microscopy. Lysine-modified lipids form stable LNPs with siRNAs and induced significant gene silencing activity; thus, their cellular uptake and endosomal escape capability were compared. Cy5-labeled IKKa siRNA was entrapped in LNPs to monitor cellular uptake (FIG. 6A). The percentage of cells that take up the LNPs did not show a significant difference among the four LNPs (FIG. 6B). However, the amounts of the Cy5-labeled IKKα siRNA in the cells are different. As illustrated in FIG. 6C, the dye intensity in the cells showed a clear correlation with the gene silencing activity of the LNPs. These data suggested that the LHHK LNPs can deliver the maximum amount of siRNA into the cells compared to the other LNPs.

Subsequently, confocal microscopy was carried out to study the intracellular distribution of the LNPs in PC3 cells (FIG. 6D, 6E). The results were consistent with the flow cytometry analysis of cellular uptake. All cells showed cellular uptake for all formulations; however, the amount of uptake in the cells differed for each LNP, suggesting a difference in cellular uptake as a percentage versus the dye intensity in the cells. In addition, It has been reported that the majority of LNPs are trapped inside the lysosome although a very low amount of successful siRNA delivery into the cytoplasm produces effective gene silencing [5]. Thus, to analyze the endosomal release capability and intracellular distribution of siRNAs, Pearson's correlation coefficient of 4-hour confocal images of cellular uptake study was calculated using ImageJ software to study the correlation between Cy5-labeled IKKa siRNA (red) and Lysotracker Red (green) (FIG. 6F). The results suggested that all LNPs have a moderate correlation at 4 h. Most importantly, LHHK had the lowest Pearson's coefficient, suggesting that it has the highest endosomal escape capability compared to the other lipids. This result demonstrated that the LNPs were able to induce endosomal escape and release the siRNAs into the cytoplasm, which is the rate-limiting step of gene silencing.

A possible mechanism for the highest intracellular delivery of LHHK LNP is that the degree of lipid ionization at the endosomal pH is critical for silencing activity. For example, LK LNP showed limited ionization at the late endosomal pH 5.2 (FIGS. 4C and 4E), which explains its low silencing activity (FIG. 4A). As histidine moiety was added to LK, the transfection effect was improved (FIG. 4A). LHHK (pKa 6.08) with two histidines and one lysine headgroup has the highest extent of ionization at endosomal pH, which helps endosomal disruption and intracellular siRNA delivery. The TNS fluorescence intensity of LNPs at late endosomal pH 5.2 demonstrated that LHHK LNPs has the maximum ionization (FIG. 4E). This result is consistent with a previous report showing the high degree of surface ionization at late endosomal pH enhances the potency of LNPs [34].

### Gene Silencing and Cell Proliferation Study

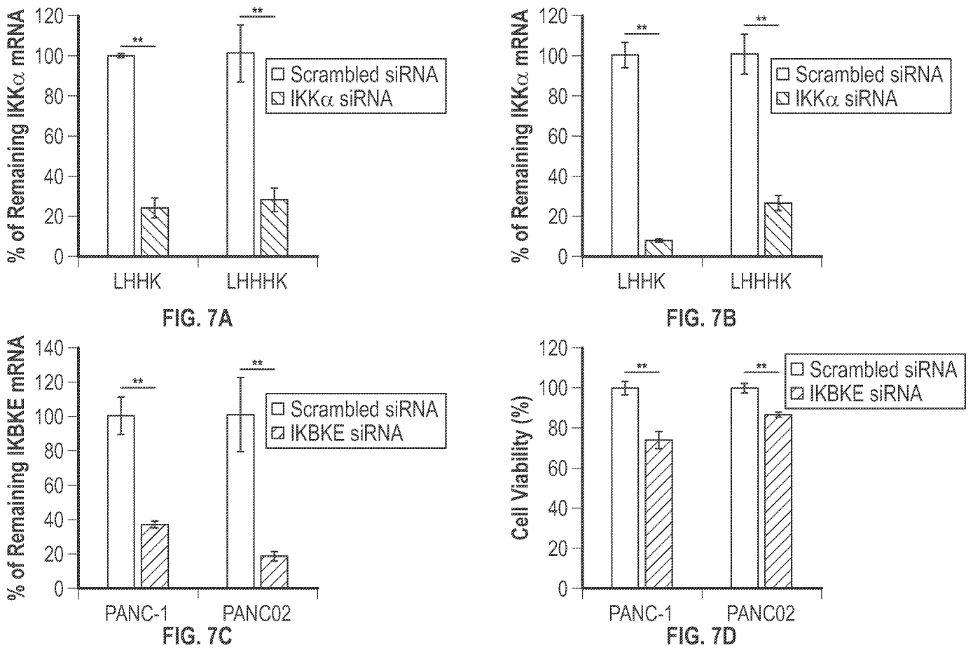

IKKa silencing in PC3 and DU 145 prostate cancer cells with the two best LNPs, i.e., LHHK and LHHHK (FIG. 7A, 7B) were evaluated. The IKKα mRNA level was measured using RT-PCR to analyze gene silencing with IKKα siRNA. The extent of gene silencing observed with both LNPs in both cell lines confirms that the LNPs are efficient for transfecting different cell lines for siRNA delivery. However, we previously observed that silencing IKKα does not inhibit the cell proliferation rate. Thus, it is not possible to see the cell proliferation effect with the IKKα silencing in prostate cancer cells. Meanwhile, we wanted to see the LNPs were effective for different strains, various cell lines, and different genes.

Earlier, an IKBKE siRNA and the a delivery system for breast cancer were developed [14, 17]. That combination produces an anti-tumor effect in breast cancer and it is interesting to see whether IKBKE therapy works for other cancers. Therefore, to further explore the applications of the LNPs, whether the LHHK LNPs can silence IKBKE in pancreatic cancer cells and decrease cell proliferation was studied. As shown in FIG. 7C, the LHHK LNPs produced significant gene silencing in PANC-1 and PANC02 cells. In addition, a cell proliferation assay suggested that IKBKE silencing in pancreatic cancer is able to produce an antitumor effect by deaccelerating the cell proliferation rate (FIG. 7D). In summary, this information illustrates that LHHK LNPs are able to deliver siRNA to various cancer cells to treat cancer.

### In Vivo Anti-Tumor Activity and Safety Assay

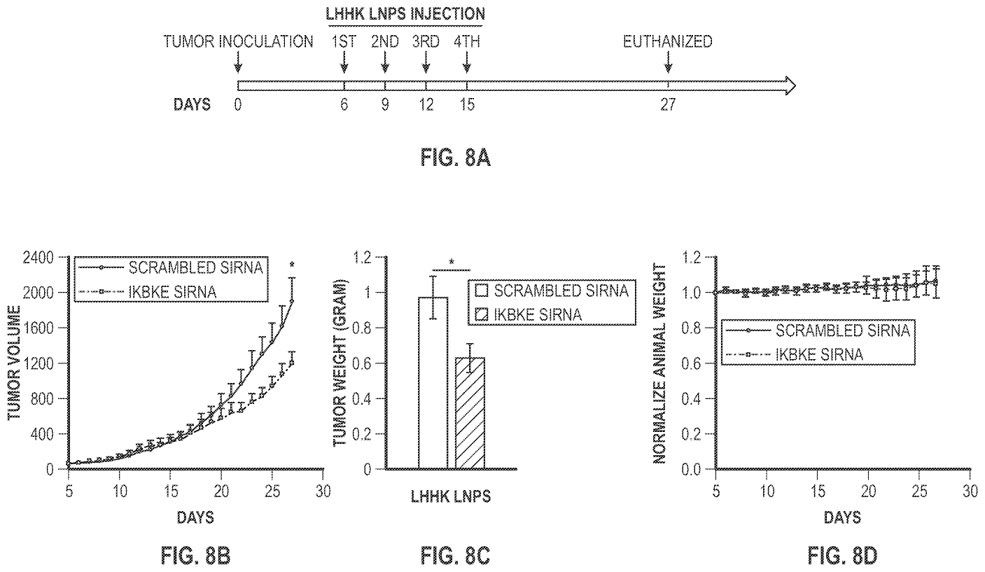

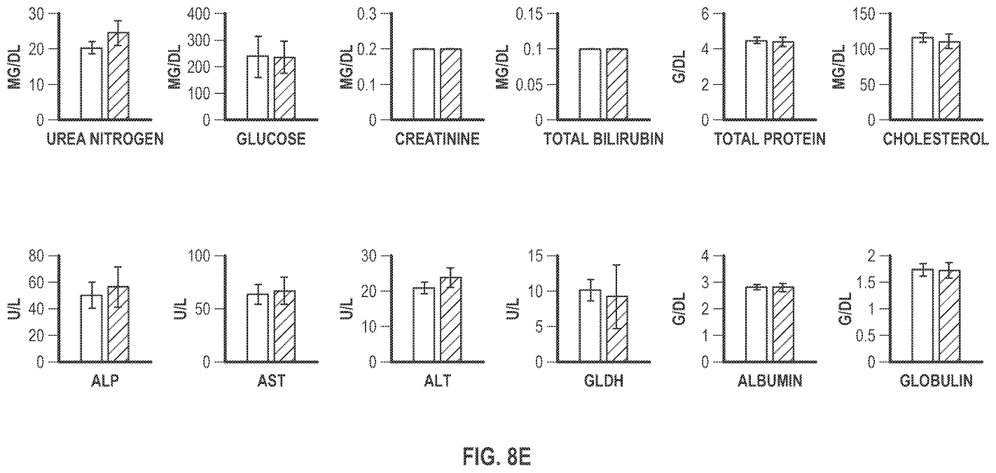

To evaluate the in vivo activity of the LHHK LNP, IKBKE siRNA was loaded into the LNP and administrated around the pancreatic tumor in a mouse model. Scramble siRNA loaded LNP was used as the control. Both LNPs were injected four times on day 6, 9, 12, and 15 (FIG. 8A). As shown in FIGS. 8B and 8C, Tumor growth was significantly suppressed in the IKBKE siRNA treated group. The tumor inhibition effect is consistent with the in vitro anti-proliferation effect of the IKBKE siRNA LNP in PANC02 cells (FIG. 7D). These results also confirms the functional importance of IKBKE in pancreatic cancer progression [35].

Body weight was monitored every day, and both groups exhibited steady body weight during the treatment (FIG. 8D). Biochemical analysis of the plasma showed no difference between the IKBKE siRNA and scramble siRNA treated group, suggesting a good safety of the IKBKE siRNA (FIG. 8E). In particular, there was no elevation of Alanine transaminase (ALT) and Aspartate Aminotransferase (AST). By contrast, liver toxicity is a common side effect of lipid-based delivery systems. Overall, these data suggest a good safety of the LHHK LNP in the mouse model.

LNPs represent a highly advanced delivery system and have been used widely in nucleic acid delivery. From a therapeutic perspective, the nanomaterials typically used for RNAi offer a narrow toxicity window. The introduction of biodegradability into the LNPs is a method of minimizing the toxicity associated with unnatural lipid molecules. In addition, the introduction of natural amino acids in the design of LNPs makes them more biocompatible.

A new class of amino acid-modified lipids for siRNA delivery has been demonstrated. Lysine and histidine were used in the development of lipid nanoparticles with multiple amide bonds to enhance biodegradability and biocompatibility. The LHHK LNPs are safe and silence target genes in different types of cancer cells. The pKa value of 6.08 for LHHK suggests that it ionizes in the endosome and is able to release siRNA intracellularly. In addition, LHHK LNP-based IKBKE siRNA inhibits tumor growth in a pancreatic cancer mouse model. In summary, the LHHK LNP is a novel candidate for siRNA delivery.

### Methods and Materials

2-(Aminomethyl) butanedioic acid hydrochloride was purchased from Enamine (Monmouth Jct., NJ). Dimethyl Formamide (DMF), Di-tert-butyl dicarbonate (BOC anhydride), Triethylamine (TEA), 1-[Bis(dimethylamino)methylene]-1H-1,2,3-triazolo[4,5-b]pyridinium 3-oxide hexafluorophosphate (HATU), N,N-Diisopropylethylamine (DIPEA), trifluoroacetic acid (TFA) N,N′-Di-Boc-L-histidine dicyclohexylammonium salt, Nalpha,Nepsilon-Di-Boc-L-lysine and cholesterol were purchased from Fischer Scientific (Pittsburgh, PA). 1,2-Distearoyl-sn-glycero-3-phosphoethanolamine-poly(ethylene glycol) (DSPE-mPEG (2000)) was purchased from Nanocs Inc (New York, NY). 1,2-dipalmitoyl-sn-glycero-3-phosphocholine (DPPC) was purchased from Avanti Polar Lipids. Luciferase siRNA (5′-CUUACGCUGAGUACUUCGAtt-3′ (SEQ ID NO: 3)), human IKKα siRNA (5′-GGAGAUCUCCGAAAGCUGCtt-3′ (SEQ ID NO: 4)), human IKBKE siRNA (5′-GGUCUUCAACACUACCAGCtt-3′ (SEQ ID NO: 5)), mouse IKBKE siRNA (5′-GGUCUUCAACUCAGCCAGCtt-3 (SEQ ID NO: 6)) and Lipofectamine® 2000 were ordered from Invitrogen (Carlsbad, CA). 2-(p-toluidino)-6-naphthalene sulfonic acid (TNS) was obtained from Sigma Aldrich. CellTiter-Glo luminescent cell viability assay kit and ONE-Glo™ luciferase assay system were purchased from Promega (Madison, WI)

### Cell Culture

PC3, DU145 and PANC-1 cells were purchased from American Type Culture Collection (Manassas, VA). Panc02 cell line was kindly provided by Dr. Gregory Lesinski at Emory University. PC3-Luc cell line was purchased from Sekisui XenoTech (Kansas City, KS). PC3, PANC02 and PC3-Luc cells were cultured in RPMI with 10% fetal bovine serum (FBS), 1% streptomycin, and 1% penicillin. DU145 and PANC-1 cells were cultured in DMEM medium with 10% FBS, 1% streptomycin, and 1% penicillin. The cells were incubated at 37° C. in a humidified atmosphere with 5% CO2.

### Synthesis and Characterization of the Lipid Library (Scheme 1)

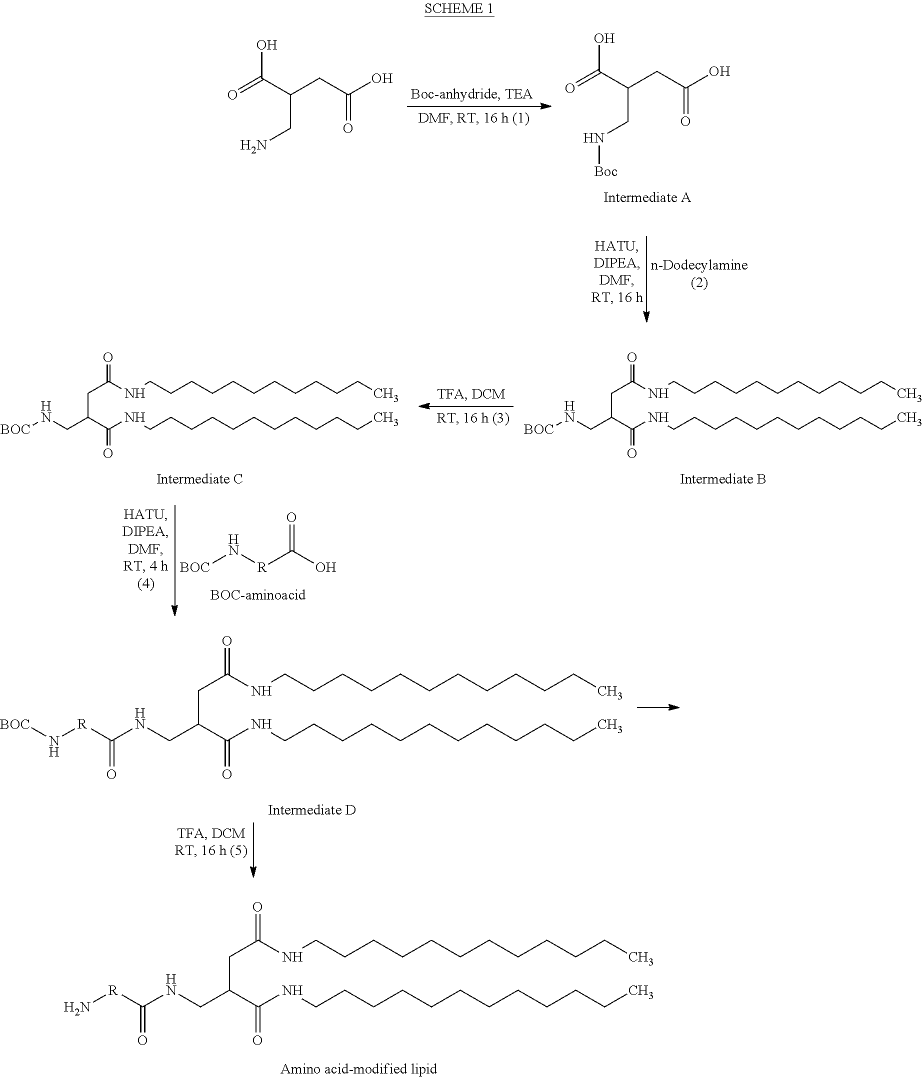

Synthetic scheme for the amino acid-modified lipids. The lipids were synthesized using 2-(aminomethyl) butanedioic acid, dodecyl amine, and Boc-protected amino acids. The reagents and reaction conditions are illustrated for each step. Steps 4 and 5 were repeated for the addition of each amino acid in the headgroup.

2-(Aminomethyl) butanedioic acid hydrochloride was dissolved in DMF and reacted with BOC-anhydride in the presence of TEA for 16 h. After the reaction was complete, the product was dried under vacuum and triturated with diethyl ether to obtain ‘Intermediate A’. The BOC-protected ‘Intermediate A’ was reacted with dodecyl amine in DMF with acid-amine coupling reagents (HATU and DIPEA). After 16 h, the reaction mixture was added to water, and ‘Intermediate B’ was extracted with ethyl acetate, which was dried under vacuum to obtain crude materials. The crude material was suspended in methanol, and the residue of pure ‘Intermediate B’ was collected after filtration. Later, ‘Intermediate B’ was reacted with TFA in DCM for 16 h. The reaction mixture was dried under vacuum, and the crude was suspended in 5 M NaOH solution and extracted with DCM to obtain ‘Intermediate C’ in free amine form.

The BOC-protected amino acid was reacted with ‘Intermediate C’ and deprotected as described above to prepare amino acid-modified lipids. Multiple amino acid-modified lipids were synthesized by repeating steps 4 and 5. The lipids were purified by cyano-modified silica column using CombiFlash and characterized by LC/MS and 1HNMR spectroscopy. The 1HNMR analysis of lipids was carried out on a 400 MHz NMR (Bruker) in methanol-d4. Additionally, the molecular weight of the compound was confirmed with a 3200 Q-trap Lc/MS (Sciex) system. The detailed procedure is described in the supporting material.

### Preparation of LNPs

Lipid nanoparticles were prepared by mixing a solution of lipids in ethanol with siRNA in 50 mM citrate buffer (pH 3) at a 7-to-1 weight ratio of amino acid-modified lipid to siRNA. Briefly, synthesized ionizable lipids, DSPE mPEG, DPPC, and cholesterol were dissolved in ethanol and mixed at a molar ratio of 55:2:10:33. LNPs were prepared by mixing siRNA in citrate buffer solution with a ½ volume of ethanol-containing lipids. The LNP solution was dialyzed against PBS for 2 h to remove ethanol.

### Characterization of LNPs

The size, polydispersity index (PDI), and zeta potential of the LNPs were measured by a Zetasizer Nano ZS (Malvern Instruments, Westborough, MA). Additionally, the size and morphology of the LNPs were studied using transmission electron microscopy (TEM).

The siRNA entrapment for LNPs was measured with the agarose gel retardation assay. In brief, a 2% agarose gel was prepared with GelRed™, and the LNPs and free siRNA samples were loaded. The gel was run in TBE buffer at 70 V for 30 minutes. Images of the gel were taken with GelDoc (Bio-Rad, Hercelus, CA).

### pKa of LNPs

A TNS fluorescence assay was used to measure the apparent pKa of the LNPs. A series of buffers ranging from pH 2 to 10.4 were prepared using 10 mM citrate, 10 mM borate, 10 mM Phosphate, and 159 mM NaCl. Blank LNPs were prepared and diluted in each buffer solution to 20 μM. Two microliters of TNS in DMSO (300 μM) was added to each well of a 96 well-plate and mixed with 100 μL of a series of buffer solutions containing LNPs. The plate was stirred for 10 minutes, and fluorescence was measured using a plate reader (SpectraMax M5e, Molecular Devices, LLC., San Jose, CA). The pH of the half-maximum fluorescence is the apparent pKa of LNPs.

### Luciferase Silencing

PC3-Luc cells (1.2>104) were seeded in a 96-well plate. After 18 h of incubation, luciferase siRNA and scrambled siRNA-loaded LNPs were added to 100 μL of Opti-MEM to achieve a final concentration of 100 nM siRNA. After 5 h, an equal volume of 10% serum-containing culture medium was added to each well. After 48 h of transfection, the volume of the medium was adjusted to 100 μL and mixed with an equal volume of a ONE-Glo™ Luciferase Assay solution. After 10 minutes, luminescence was measured with a plate reader to quantify luciferase silencing.

### Cellular Cytotoxicity

PC3-Luc cells (1.2×104) were seeded in a 96-well plate and transfected with scrambled siRNA-loaded LNPs after 18 h. At different transfection time points, an equal volume of CellTiter-Glo solution was added to the wells and mixed for 10 minutes. Then, luminescence values were measured with a plate reader to calculate cell viability.

Cellular Uptake with Flow Cytometry and Confocal Microscopy

A total of 2.5×105 PC3 cells were seeded in 6-well plates 18 h before transfection. Cy5-labeled IKKα siRNA-loaded LNPs of lipids (LK, LHK, LHHK, and LHHHK) and free Cy5-labeled IKKα siRNA were added to the wells with Opti-MEM™ at a concentration of 100 nM siRNA. Then, the cells were incubated for 1, 2, and 4 h at 37° C. and washed with heparin (1 mg/mL) to remove nonspecifically bound particles. The cells were detached with 0.25% trypsin and washed with DPBS, and then cellular uptake was analyzed with a FACS II flow cytometer (BD Instrument, NJ). For confocal microscopy, 5×104 PC3 cells were seeded in a four-well chamber slide and transfected with the LNPs for 2 and 4 h. The cells were then incubated with 250 nM LysoTracker Red for 1 h, fixed with 10% formalin, and studied with a confocal microscope (Leica TCS SP5, Germany).

### Hemolysis Assay

Fresh human red blood cells (RBCs) were washed twice with PBS (pH 7.4) and diluted in either pH 7.4 or pH 5.6 phosphate buffer to achieve a 2% (v/v) RBC solution. In a v-bottom 96-well plate, 20 μL of blank LNPs was added to 180 μL of the 2% (v/v) RBC solution in both buffer solutions. After incubation at 37° C. for 1 h, the plate was centrifuged at 1,000×g for 5 min at 4° C., and 100 μL of the supernatant from each well was transferred into a new 96-well plate to read UV absorption at 540 nm. Negative and positive control experiments were carried out with buffer alone and 1% Triton X-100, respectively.

### Serum Stability

LHHK siRNA LNPs were mixed with 50% human serum and incubated at 37° C. for 1, 6, and 24 h. The samples were then incubated with heparin for 30 min on ice to release siRNAs, separated on a 2% agarose gel, and visualized with GelRed staining.

### IKKA and IKBKE Gene Silencing

IKKα gene silencing was evaluated in prostate cancer PC3 and DU145 cells. Briefly, PC3 cells (3.5×104 per well) or DU145 cells (5×104 per well) were seeded in 12-well plates. After 18 h of incubation, IKKα siRNA-loaded LNPs of LHHK and LHHHK were added to the cells at a concentration of 100 nM. After 24 h, total RNA was extracted, and the IKKα mRNA level was quantified by RT-PCR. 18S rRNA was used as the internal control in RT-PCR. Similar protocol was used to study IKBKE silencing in pancreatic cancer PANC-1 (5×104 per well) and PANC02 (5×104 per well) cells. A scrambled siRNA was used as negative control.

### Cell Proliferation Assay

PANC-1 and PANC02 cells (5×103) were seeded in 96-well plates. After 18 h of incubation, IKBKE siRNA-loaded LHHK LNPs were transfected at a concentration of 100 nM. Cell viability was measured with CellTiter-Glo solution after 96 h.

### In Vivo Anti-Tumor Activity

The animal protocol was approved by the Institutional Animal Care and Use Committee (IACUC) of the University of Missouri-Kansas City. PANC02 cells (1×106) were mixed with matrigel at a ratio of 1:1 (v/v) and implanted subcutaneously into the back of C57BL/6 male mice. Once tumor size reached ˜75 mm3, the mice were randomly assigned into two groups for the treatment with IKBKE siRNA and scramble siRNA encapsulated in LHHK LNPs. The LNPs were administered around the tumor at 0.4 mg/kg on days 6, 9, 12, and 15. Tumor volumes and body weights were monitored daily. Tumor volumes were calculated using the formula: tumor volume=(length×width2)/2. At the end of the study, the plasma was collected for biochemical analysis at the University of Missouri Veterinary Medical Diagnostic Laboratory.

### Statistical Analysis

Data are presented as the mean±standard deviation (SD). Statistical analysis of multiple groups was performed using a one-way analysis of variance (ANOVA) with Tukey's post hoc test. The T-test was applied to analyze the difference between two groups. The following p-values were considered to be significant: * p<0.05, ** p<0.01

### REFERENCES

[1] R. L. Setten, J. J. Rossi, S. P. Han, The current state and future directions of RNAi-based therapeutics, Nature reviews. Drug discovery 18 (6) (2019) 421-446.
[2] R. Kanasty, J. R. Dorkin, A. Vegas, D. Anderson, Delivery materials for siRNA therapeutics, Nature materials 12 (11) (2013) 967-77.
[3] J. A. Kulkarni, P. R. Cullis, R. van der Meel, Lipid Nanoparticles Enabling Gene Therapies: From Concepts to Clinical Utility, Nucleic acid therapeutics 28 (3) (2018) 146-157.
[4] S. Rietwyk, D. Peer, Next-Generation Lipids in RNA Interference Therapeutics, ACS nano 11 (8) (2017) 7572-7586.
[5] G. Sahay, W. Querbes, C. Alabi, A. Eltoukhy, S. Sarkar, C. Zurenko, E. Karagiannis, K. Love, D. Chen, R. Zoncu, Y. Buganim, A. Schroeder, R. Langer, D. G. Anderson, Efficiency of siRNA delivery by lipid nanoparticles is limited by endocytic recycling, Nature biotechnology 31 (7) (2013) 653-8.
[6] S. C. Semple, A. Akinc, J. Chen, A. P. Sandhu, B. L. Mui, C. K. Cho, D. W. Sah, D. Stebbing, E. J. Crosley, E. Yaworski, I. M. Hafez, J. R. Dorkin, J. Qin, K. Lam, K. G. Rajeev, K. F. Wong, L. B. Jeffs, L. Nechev, M. L. Eisenhardt, M. Jayaraman, M. Kazem, M. A. Maier, M. Srinivasulu, M. J. Weinstein, Q. Chen, R. Alvarez, S. A. Barros, S. De, S. K. Klimuk, T. Borland, V. Kosovrasti, W. L. Cantley, Y. K. Tam, M. Manoharan, M. A. Ciufolini, M. A. Tracy, A. de Fougerolles, I. MacLachlan, P. R. Cullis, T. D. Madden, M. J. Hope, Rational design of cationic lipids for siRNA delivery, Nature biotechnology 28 (2) (2010) 172-6.
[7] S. Patel, J. Kim, M. Herrera, A. Mukherjee, A. V. Kabanov, G. Sahay, Brief update on endocytosis of nanomedicines, Advanced drug delivery reviews 144 (2019) 90-111.
[8] K. J. Kauffman, J. R. Dorkin, J. H. Yang, M. W. Heartlein, F. DeRosa, F. F. Mir, O. S. Fenton, D. G. Anderson, Optimization of Lipid Nanoparticle Formulations for mRNA Delivery in Vivo with Fractional Factorial and Definitive Screening Designs, Nano letters 15 (11) (2015) 7300-6.
[9] S. Ramishetti, I. Hazan-Halevy, R. Palakuri, S. Chatterjee, S. Naidu Gonna, N. Dammes, I. Freilich, L. Kolik Shmuel, D. Danino, D. Peer, A Combinatorial Library of Lipid Nanoparticles for RNA Delivery to Leukocytes, Advanced materials 32 (12) (2020) e1906128.
[10] M. A. Maier, M. Jayaraman, S. Matsuda, J. Liu, S. Barros, W. Querbes, Y. K. Tam, S. M. Ansell, V. Kumar, J. Qin, X. Zhang, Q. Wang, S. Panesar, R. Hutabarat, M. Carioto, J. Hettinger, P. Kandasamy, D. Butler, K. G. Rajeev, B. Pang, K. Charisse, K. Fitzgerald, B. L. Mui, X. Du, P. Cullis, T. D. Madden, M. J. Hope, M. Manoharan, A. Akinc, Biodegradable lipids enabling rapidly eliminated lipid nanoparticles for systemic delivery of RNAi therapeutics, Molecular therapy: the journal of the American Society of Gene Therapy 21 (8) (2013) 1570-8.
[11] Z. R. Lu, V. E. A. Laney, R. Hall, N. Ayat, Environment-Responsive Lipid/siRNA Nanoparticles for Cancer Therapy, Advanced healthcare materials 10 (5) (2021) e2001294.
[12] C. Lorenzer, M. Dirin, A. M. Winkler, V. Baumann, J. Winkler, Going beyond the liver: progress and challenges of targeted delivery of siRNA therapeutics, Journal of controlled release: official journal of the Controlled Release Society 203 (2015) 1-15.
[13] R. Mahato, B. Qin, K. Cheng, Blocking IKKalpha expression inhibits prostate cancer invasiveness, Pharmaceutical research 28 (6) (2011) 1357-69.
[14] B. Qin, K. Cheng, Silencing of the IKKepsilon gene by siRNA inhibits invasiveness and growth of breast cancer cells, Breast Cancer Res 12 (5) (2010) R74.
[15] B. Qin, Z. Chen, W. Jin, K. Cheng, Development of cholesteryl peptide micelles for siRNA delivery, Journal of controlled release: official journal of the Controlled Release Society 172 (1) (2013) 159-168.
[16] Y. Li, Z. Zhao, C. Y. Lin, Y. Liu, O. K. F. Staveley, G. Li, K. Cheng, Silencing PCBP2 normalizes desmoplastic stroma and improves the antitumor activity of chemotherapy in pancreatic cancer, Theranostics 11 (5) (2021) 2182-2200.
[17] Z. Zhao, Y. Li, H. Liu, A. Jain, P. V. Patel, K. Cheng, Co-delivery of IKBKE siRNA and cabazitaxel by hybrid nanocomplex inhibits invasiveness and growth of triple-negative breast cancer, Sci Adv 6 (29) (2020) eabb0616.
[18] A. Singh, P. Trivedi, N. K. Jain, Advances in siRNA delivery in cancer therapy, Artificial cells, nanomedicine, and biotechnology 46 (2) (2018) 274-283.
[19] X. Hou, T. Zaks, R. Langer, Y. Dong, Lipid nanoparticles for mRNA delivery, Nat Rev Mater 6 (12) (2021) 1078-1094.
[20] M. Qiu, Z. Glass, J. Chen, M. Haas, X. Jin, X. Zhao, X. Rui, Z. Ye, Y. Li, F. Zhang, Q. Xu, Lipid nanoparticle-mediated codelivery of Cas9 mRNA and single-guide RNA achieves liver-specific in vivo genome editing of Angpt13, Proc Natl Acad Sci USA 118 (10) (2021).
[21] X. Zhao, J. Chen, M. Qiu, Y. Li, Z. Glass, Q. Xu, Imidazole-Based Synthetic Lipidoids for In Vivo mRNA Delivery into Primary T Lymphocytes, Angew Chem Int Ed Engl 59 (45) (2020) 20083-20089.
[22] F. Ma, L. Yang, Z. Sun, J. Chen, X. Rui, Z. Glass, Q. Xu, Neurotransmitter-derived lipidoids (NT-lipidoids) for enhanced brain delivery through intravenous injection, Sci Adv 6 (30) (2020) eabb4429.
[23] X. Zhao, Z. Glass, J. Chen, L. Yang, D. L. Kaplan, Q. Xu, mRNA Delivery Using Bioreducible Lipidoid Nanoparticles Facilitates Neural Differentiation of Human Mesenchymal Stem Cells, Adv Healthc Mater 10 (4) (2021) e2000938.
[24] A. Akinc, A. Zumbuehl, M. Goldberg, E. S. Leshchiner, V. Busini, N. Hossain, S. A. Bacallado, D. N. Nguyen, J. Fuller, R. Alvarez, A. Borodovsky, T. Borland, R. Constien, A. de Fougerolles, J. R. Dorkin, K. Narayanannair Jayaprakash, M. Jayaraman, M. John, V. Koteliansky, M. Manoharan, L. Nechev, J. Qin, T. Racie, D. Raitcheva, K. G. Rajeev, D. W. Sah, J. Soutschek, I. Toudjarska, H. P. Vornlocher, T. S. Zimmermann, R. Langer, D. G. Anderson, A combinatorial library of lipid-like materials for delivery of RNAi therapeutics, Nat Biotechnol 26 (5) (2008) 561-9.
[25] J. Liu, J. Chang, Y. Jiang, X. Meng, T. Sun, L. Mao, Q. Xu, M. Wang, Fast and Efficient CRISPR/Cas9 Genome Editing In Vivo Enabled by Bioreducible Lipid and Messenger RNA Nanoparticles, Adv Mater 31 (33) (2019) e1902575.
[26] Q. Xu, H. Deng, X. Li, Z. S. Quan, Application of Amino Acids in the Structural Modification of Natural Products: A Review, Front Chem 9 (2021) 650569.
[27] L. Cynober, D. M. Bier, P. Stover, M. Kadowaki, S. M. Morris, R. Elango, M. Smriga, Proposals for Upper Limits of Safe Intake for Methionine, Histidine, and Lysine in Healthy Humans, J Nutr 150 (Suppl 1) (2020) 2606S-2608S.
[28] K. A. Whitehead, J. R. Dorkin, A. J. Vegas, P. H. Chang, O. Veiseh, J. Matthews, O. S. Fenton, Y. Zhang, K. T. Olejnik, V. Yesilyurt, D. Chen, S. Barros, B. Klebanov, T. Novobrantseva, R. Langer, D. G. Anderson, Degradable lipid nanoparticles with predictable in vivo siRNA delivery activity, Nature communications 5 (2014) 4277.
[29] L. Li, D. Zahner, Y. Su, C. Gruen, G. Davidson, P. A. Levkin, A biomimetic lipid library for gene delivery through thiol-yne click chemistry, Biomaterials 33 (32) (2012) 8160-6.
[30] E. Blanco, H. Shen, M. Ferrari, Principles of nanoparticle design for overcoming biological barriers to drug delivery, Nature biotechnology 33 (9) (2015) 941-51.
[31] P. Patel, N. M. Ibrahim, K. Cheng, The Importance of Apparent pKa in the Development of Nanoparticles Encapsulating siRNA and mRNA, Trends in pharmacological sciences (2021).
[32] C. A. Alabi, K. T. Love, G. Sahay, H. Yin, K. M. Luly, R. Langer, D. G. Anderson, Multiparametric approach for the evaluation of lipid nanoparticles for siRNA delivery, Proceedings of the National Academy of Sciences of the United States of America 110 (32) (2013) 12881-6.
[33] B. C. Evans, C. E. Nelson, S. S. Yu, K. R. Beavers, A. J. Kim, H. Li, H. M. Nelson, T. D. Giorgio, C. L. Duvall, Ex vivo red blood cell hemolysis assay for the evaluation of pH-responsive endosomolytic agents for cytosolic delivery of biomacromolecular drugs, Journal of visualized experiments: JoVE (73) (2013) e50166.
[34] K. A. Hajj, R. L. Ball, S. B. Deluty, S. R. Singh, D. Strelkova, C. M. Knapp, K. A. Whitehead, Branched-Tail Lipid Nanoparticles Potently Deliver mRNA In Vivo due to Enhanced Ionization at Endosomal pH, Small 15 (6) (2019) e1805097.
[35] M. Rajurkar, K. Dang, M. G. Fernandez-Barrena, X. Liu, M. E. Fernandez-Zapico, B. C. Lewis, J. Mao, IKBKE Is Required during KRAS-Induced Pancreatic Tumorigenesis, Cancer Res 77 (2) (2017) 320-329.

1.-32. (canceled)

33. A compound having formula I 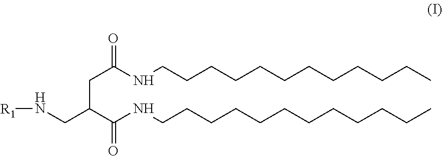
wherein R¹ is a peptide of sequence (AA₁)ₐ-(AA₂)_{b} connected via the C-terminus of the peptide to the lipid moiety 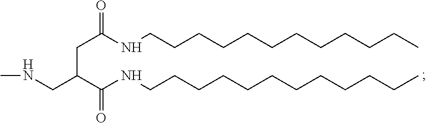 [SMILES: CCCCCCCCCCCCNC(=O)CC(CNC)C(=O)NCCCCCCCCCCCC ; ";"]
AA₁ is selected from the group consisting of 2,3-diaminopropanoic acid, 2,4-diaminobutanoic acid, ornithine, lysine, 2,7-diaminoheptanoic acid, 2,8-diaminooctanoic acid, 2-amino-3-guanidinopropanoic acid, 2-amino-4-guanidino-butanoic acid and 2-amino-6-guanidinopentanoic acid, and arginine, each of which can be in the 2R, 2S, or racemic form;
AA₂ is a histidine in the 2R, 2S, or racemic form; and
a is 0, or 1 and b is 0, 1, 2, 3, or 4 where at least one of a or b is not 0.

34. The compound of claim 33 wherein one of:
AA₁ is lysine,
AA₂ is histidine, or
AA₁ is lysine and AA₂ is histidine.

35. The compound of claim 34 wherein one of:
AA₁ is 2S-lysine,
AA₂ is 2S-histidine, or
AA₁ is 2S-lysine and AA₂ is 2S-histidine.

36. The compound of claim 33 wherein AA₁ is lysine and AA₂ is histidine, further wherein the compound is selected from the group consisting of:

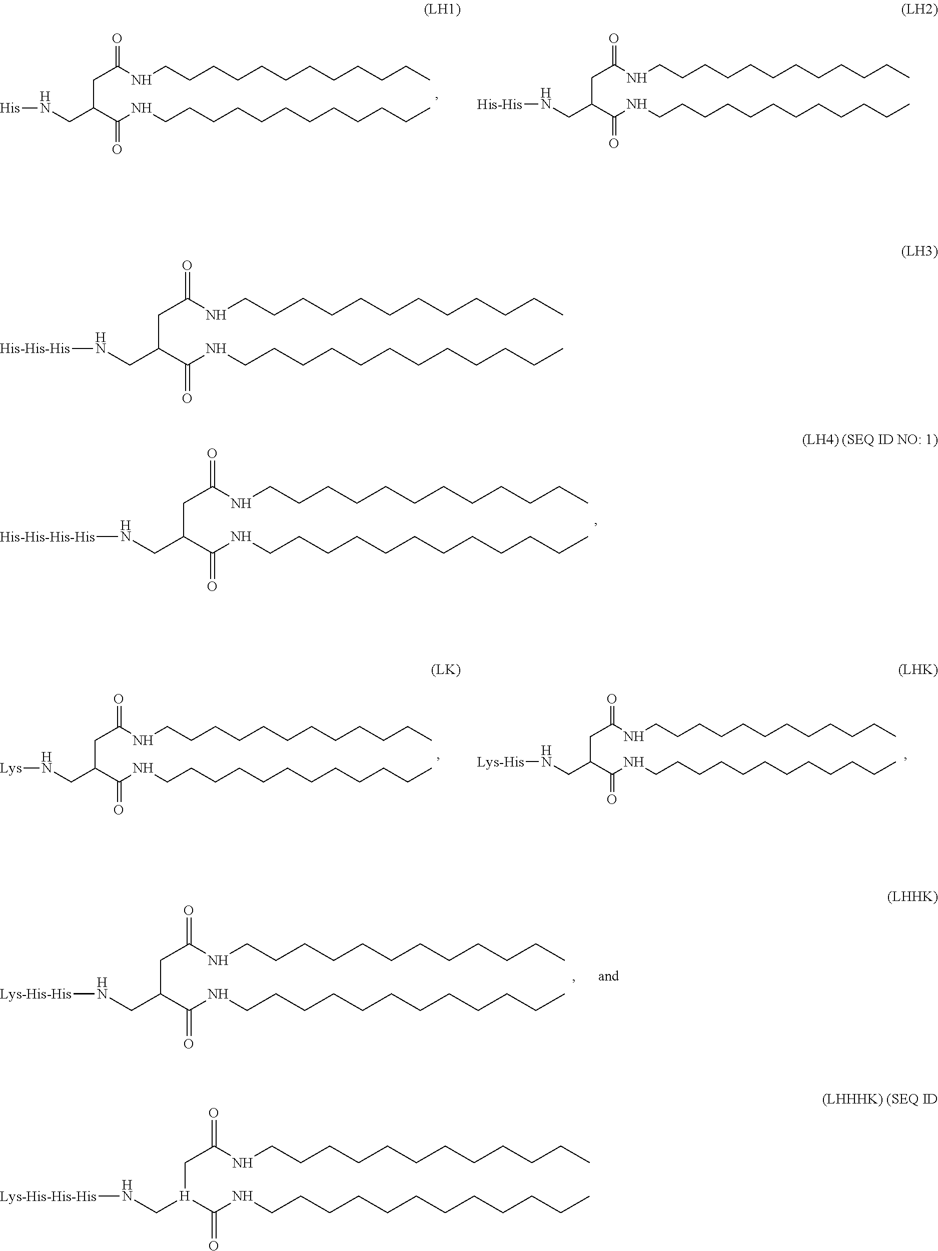

37. A lipid nanoparticle comprising one or more of a compound having formula I 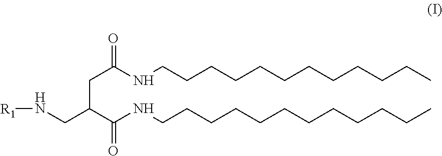
wherein R¹ is a peptide of sequence (AA₁)ₐ-(AA₂)_{b} connected via the C-terminus of the peptide to the lipid moiety 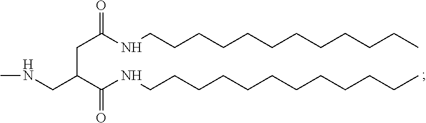 [SMILES: CCCCCCCCCCCCNC(=O)CC(CNC)C(=O)NCCCCCCCCCCCC ; ";"]
AA₁ is selected from the group consisting of 2,3-diaminopropanoic acid, 2,4-diaminobutanoic acid, ornithine, lysine, 2,7-diaminoheptanoic acid, 2,8-diaminooctanoic acid, 2-amino-3-guanidinopropanoic acid, 2-amino-4-guanidino-butanoic acid and 2-amino-6-guanidinopentanoic acid, and arginine, each of which can be in the 2R, 2S, or racemic form;
AA₂ is a histidine in the 2R, 2S, or racemic form; and
a is 0, or 1 and b is 0, 1, 2, 3, or 4 where at least one of a or b is not 0.

38. The lipid nanoparticle of claim 37 wherein one of:
AA₁ is lysine,
AA₂ is histidine, or
AA₁ is lysine and AA₂ is histidine.

39. The lipid nanoparticle of claim 38 wherein one of:
AA₁ is 2S-lysine,
AA₂ is 2S-histidine, or
AA₁ is 2S-lysine and AA₂ is 2S-histidine.

40. The lipid nanoparticle of claim 37 wherein the compound is selected from the group consisting of

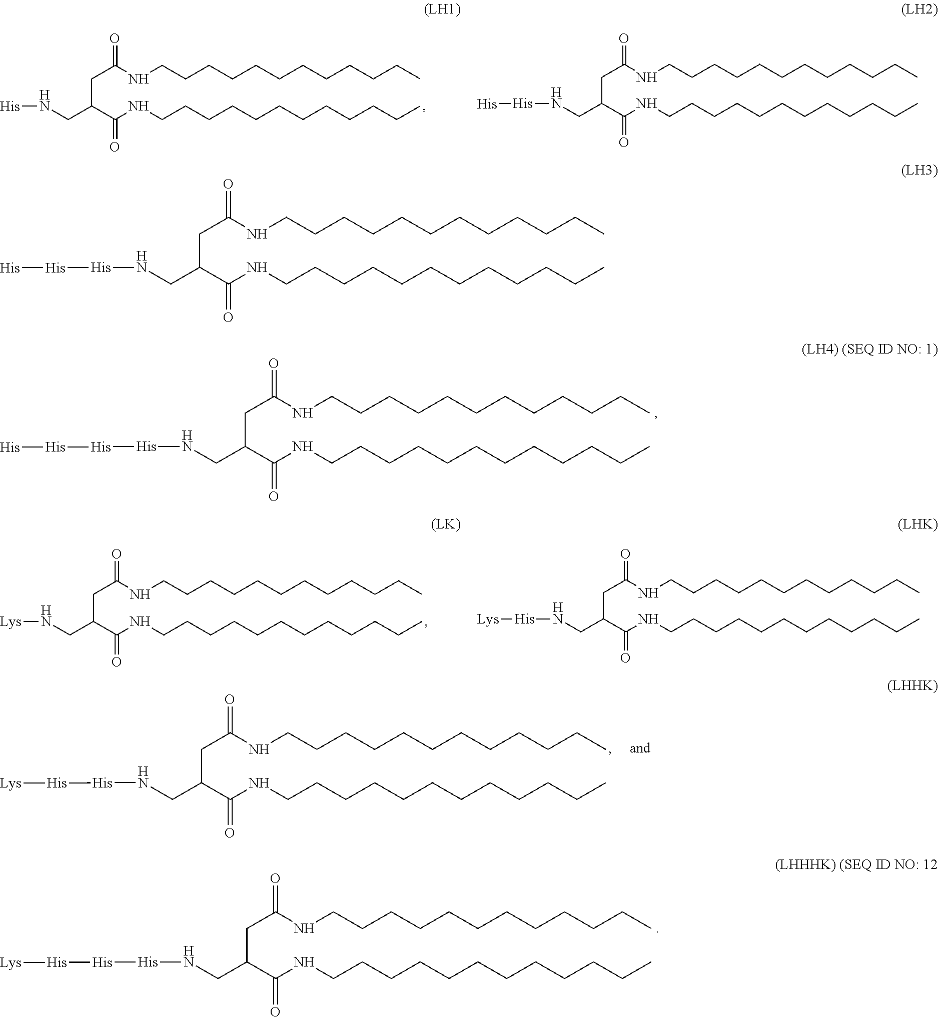

41. The lipid nanoparticle of claim 37 further comprising an oligonucleotide selected from the group consisting of siRNA, miRNA, shRNA, and mRNA.

42. The lipid nanoparticle of claim 41 wherein the oligonucleotide is a siRNA and wherein the siRNA silences expression of an overexpressed oncogene related to the progression of a cancer.

43. The lipid nanoparticle of claim 42 wherein the cancer is at least one of breast cancer, prostate cancer, or pancreatic cancer.

44. A method of treating a cancer patient, the method comprising the use of a lipid nanoparticle comprising one or more of a compound having formula I 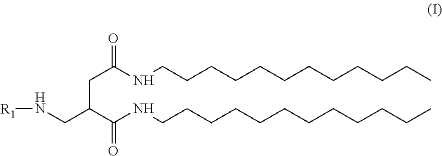
wherein R¹ is a peptide of sequence (AA₁)ₐ-(AA₂)_{b} connected via the C-terminus of the peptide to the lipid moiety 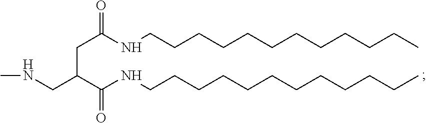 [SMILES: CCCCCCCCCCCCNC(=O)CC(CNC)C(=O)NCCCCCCCCCCCC ; ";"]
AA₁ is selected from the group consisting of 2,3-diaminopropanoic acid, 2,4-diaminobutanoic acid, ornithine, lysine, 2,7-diaminoheptanoic acid, 2,8-diaminooctanoic acid, 2-amino-3-guanidinopropanoic acid, 2-amino-4-guanidino-butanoic acid and 2-amino-6-guanidinopentanoic acid, and arginine, each of which can be in the 2R, 2S, or racemic form;
AA₂ is a histidine in the 2R, 2S, or racemic form; and
a is 0, or 1 and b is 0, 1, 2, 3, or 4 where at least one of a or b is not 0,
the lipid nanoparticle further comprising a siRNA wherein the siRNA silences expression of an overexpressed oncogene related to the progression of the cancer.

45. The method of claim 44 further comprising treating a cancer patient having at least one of:
breast cancer,
prostate cancer, or
pancreatic cancer.
